(19) United States
(12) Patent Application Publication
(10) Pub. No.: US 2009/0112049 A1
(43) Pub. Date: Apr. 30, 2009

(54) HEART PUMP APPARATUS AND METHOD FOR BEATING HEART SURGERY

(75) Inventor: Fayaz Ahmed, Dhahran (SA)

Correspondence Address: BRACEWELL & GIULIANI LLP, P.O. BOX 61389, HOUSTON, TX, 77208-1389, US

(73) Assignee: Saudi Arabian Oil Company, Dhahran (SA)

(21) Appl. No.: 11/927,160

(22) Filed: Oct. 29, 2007

(51) Int. Cl. A61M 1/10

(52) U.S. Cl. 600/16

(57) ABSTRACT

Apparatus for assisting a surgeon in procedures involving the heart and methods of employing such apparatus are provided. The apparatus can include a pump, and a first fluid conduit having a distal end adapted to be inserted into the superior vena cava of a beating heart, a second fluid conduit having a distal end adapted to be inserted into the inferior vena cava, and a third fluid conduit having a distal end adapted to be inserted into the pulmonary artery of the beating heart, each in liquid fluid communication with the pump, which in combination can be operatively positioned to form a closed cardiac pathway extending from the vena cavae and to the pulmonary artery to thereby convey blood collected from the vena cavae into the pulmonary artery, operatively bypassing the right side of the heart. The pump is positioned to both convey blood flow from each vena cavae and to the third fluid conduit and to provide a blood reservoir which enables the provision of manual assistance to the blood flow to the lungs when blood flow is insufficient.

### BACKGROUND OF THE INVENTION

1. Field of the Invention

The present invention relates to heart surgery systems, apparatus, and methods for performing heart surgery. More specifically, the present invention relates to apparatus to perform beating heart surgery and related methods.

2. Description of Related Art

Major heart surgery is often accomplished by procedures that require a complete cardio-pulmonary bypass (CPB), along with a complete cessation of all cardiopulmonary activity. This is typically accomplished through the use of artificial heart-lung machines and/or systems, which come in various configurations. While the average mortality rate associated with CPB procedures is relatively low, it is nonetheless associated with a complication rate that is often much higher compared to when cessation of the heart and CPB are not required. The use of CPB continues to represent a major assault on a host of body systems. For example, there is a noticeable degradation of mental faculties following such surgeries in a significant percentage of patients who undergo open-heart procedures.

Open-heart surgery generally involves cutting the sternum of the patient in order to spread the chest apart and provide access to the heart. During surgery the heart is stopped, and by the use of CPB, blood is diverted from the lungs to an artificial oxygenator. At the same time, the dramatic increase in the life expectancy of the general population has resulted in patients who are more likely to be older and in poor health, with less cardiovascular, systemic, and neurologic reserve needed to recover from the trauma caused by the use of CPB. As a consequence, inflammatory, hemostatic, endocrinologic, and neurologic stresses are tolerated to a much lesser degree by a significant number of patients today, and thus, play a more significant role in CPB-induced morbidity.

One newer system is described, for example, in U.S. Pat. No. 5,478,309 by Sweezer et al., titled “Catheter System and Method for Providing Cardiopulmonary Bypass Pump Support during Heart Surgery,” which can be used in closed heart surgery to help reduce some of the complications associated with thoracotomy or median sternotomy. Sweezer et al. describes a system of venous perfusion and arterial perfusion catheters for use in obtaining total cardiopulmonary bypass support and isolation of the heart during the performance of heart surgery. The venous perfusion catheter is positioned within the right atrium and includes a conduit which extends between the superior vena cava which can be occluded by an expander or balloon located near the atrio-caval junction and the inferior vena cava which can be occluded by a second expander or balloon positioned near the atrio-caval junction to occlude the inferior vena cava. The combination of the first and second expanders or balloons prevent entry of the de-oxygenated blood into the right atrium and thereby the right ventricle. The venous perfusion catheter is connected to an exterior oxygenator and a cardiopulmonary bypass pump, which receives blood which enters the venous perfusion catheter through inlets in the catheter to oxygenate and pump the blood. The arterial perfusion catheter is connected to the oxygenator and cardiopulmonary bypass pump to return oxygenated blood through outlets in the arterial perfusion catheter, which extends into the aorta. The arterial perfusion catheter includes a balloon or expander located adjacent the aortic valve to occlude the aorta adjacent the aortic valve. Nevertheless, such systems do not overcome the disadvantages associated with a total cardiopulmonary bypass.

The combined statistics of postoperative morbidity and mortality continue to illustrate the shortcomings of CPB. The extracorporeal shunting and artificially induced oxygenation of blood activates a system-wide roster of plasma proteins and blood components in the body including those that were designed to act locally in response to infection or injury. When these potent actors are disseminated throughout the body without normal regulatory controls, the entire body becomes a virtual battleground. The degradation of mental faculties resulting from open heart procedures is commonly attributed to cerebral arterial blockage and such emboli from debris in the blood generated by the use of CPB. The adverse hemostatic consequences of CPB also include prolonged and potentially excessive bleeding. CPB-induced platelet activation, adhesion, and aggregation also contribute to depletion in platelet number, and are further compounded by the reversibly depressed functioning of platelets remaining in circulation.

The coagulation and fibrinolytic systems both contribute to hemostatic disturbances during and following CPB. The leading cause of morbidity and disability following cardiac surgery, however, as noted above, is cerebral complications. Gaseous and solid micro and macro emboli, and less often perioperative cerebral hypoperfusion, produce neurologic effects ranging from subtle neuropsychologic deficits to fatal stroke. Advances in computed tomography, magnetic resonance imaging, ultrasound, and other imaging and diagnostic techniques have added to the understanding of these complications. With the possible exception of periopertive electroencephalography, however, these technologies do not yet permit real time surgical adjustments that are capable of preventing emboli or strokes in the making. Doppler and ultrasound evaluation of the carotid artery and ascending aorta, and other diagnostic measures, can help identify surgical patients at elevated risk for stroke and are among the growing list of pharmacologic and procedural measures for reducing that risk.

CPB also affects various endocrine systems, including the thyroid gland, adrenal medulla and cortex, pituitary gland, pancreas, and parathyroid gland. These systems are markedly affected not only by inflammatory processes, but also by physical and biochemical stresses imposed by extracorporeal perfusion. Most notably, CPB is now clearly understood to induce euthyroid-sick syndrome, which is marked by profoundly depressed triiodothyronine levels persisting for days following cardiothoracic surgery. The efficacy of hormone replacement regimens to counteract this effect is currently undergoing clinical investigation. By contrast, levels of the stress hormones epinephrine, norepinephrine, and cortisol are markedly elevated during and following CPB, and hyperglycemia is also possible.

Beating heart bypass surgery has been recognized as a desirable alternative because it has the possibility of avoiding the necessity of placing the patient on a full CPB system. The medical community is currently performing more beating heart bypass surgery in an effort to avoid the use of fill CPB. Examples of such systems include U.S. Pat. No. 6,395,026 by Aboul-Hosn et al. titled “Apparatus and Method for Beating Heart Bypass Surgery,” which describes a cannula and electric pump system that provides a supplemented or augmented flow of blood to the pulmonary artery to enable the heart to continue pumping blood in its normal fashion. The system includes a cannula extending through an incision in the wall of the right atrium and into the pulmonary artery beyond the semilunar (pulmonary) valve, and a pump configured to draw blood from within the right atrium. The system is configured to provide pulmonary blood flow around the cannula, to the extent that the heart is capable, during the lifting and manipulation of the heart during surgery, and to provide sufficient blood flow through the cannula to sustain the patient, in the event of decreased output of the heart or in the event of a dysrythmia or other interruption of pulmonary blood flow by the beating heart. The system can also include a conduit which includes a first cannula extending through an incision in the vena cava to an external inline electric pump and a second cannula extending from the pump and into an incision in the pulmonary artery to supplement and augment the beating heart blood flow provided by the main portion of the system. Examples of such systems also include U.S. Pat. No. 5,688,245 by Jarvik et al. titled “Circulatory Support System,” which describes an electric pump system which includes an inline electric axial flow pump having an inlet and an inlet conduit extending through an incision in the wall of the right ventricle and an outlet and an outlet conduit extending through an incision in a wall of the pulmonary artery beyond the semilunar (pulmonary) valve to provide at least a partial bypass of the heart so as to supplement the pumping function of the heart, to thereby enable the surgeon to perform various surgical procedures. Both of these types of beating heart bypass surgery systems, however, drain blood from within the right atrium and right ventricle to then translate the blood through the pulmonary artery using an electric pump. Thus, Applicant has recognized that neither type of system provides a bloodless field, nor manually operated pump control.

Accordingly, Applicant also has recognized that the need is increasing for an apparatus and methods and associated equipment to enhance the capability and versatility of beating heart surgery and to avoid CPB procedures in any heart surgery. Further, recognized by the Applicant is that such an apparatus and methods should provide the surgeon a bloodless field and should allow positive control of blood flow by the surgical team. Particularly, recognized by the Applicant is the need for an apparatus and methods to provide right heart support and to include a substantially bloodless field, thereby making beating heart surgery more feasible.

### SUMMARY OF THE INVENTION

In view of the foregoing, embodiments of the present invention advantageously provide an apparatus and methods to enhance the ability to perform cardiac surgery that eliminates, or at least reduces, the need for a full cardiopulmonary bypass (“CPB”), making feasible the performance of beating heart bypass surgery in a substantially bloodless field. Embodiments of the present invention also advantageously provide an apparatus and methods to enhance beating heart surgery, whereby the patient's lungs are used for blood oxygenation, to thereby avoid the need for CPB or other external blood oxygenation equipment or procedure. That is, embodiments of the present invention include an apparatus that can maintain sufficient blood flow to the lungs, and therefore, can maintain circulatory blood flow throughout the body during, for example, open-heart surgery.

Embodiments of the present invention also advantageously provide an apparatus and methods that can provide right heart support to include a substantially bloodless field, further making feasible beating heart surgery. In reference to embodiments of the present invention, the “right side” of the heart generally refers to and includes the vena cavae (superior and inferior), the right atrium, the right ventricle, the pulmonary artery, and any combination thereof. The “right side” of the heart receives deoxygenated blood and provides blood flow to the lungs for oxygenation prior to being returned to the left side of the heart to be circulated through the body of the patient.

Embodiments of the present invention advantageously can overcome many of the difficulties of prior systems, apparatus, and methods by providing the surgical team positive control of blood flow, allowing the surgical team to safely maintain good cardiac output while performing beating heart surgery. To do so, embodiments of the present invention provide an apparatus to assist in procedures involving, for example, the right side of the heart during, thoracotomic, stemotomic, or other surgery. For example, according to an embodiment of an apparatus to assist in surgical procedures, such as those involving the right side of the heart, such apparatus can include a first fluid conduit including, for example, a cannula having a distal portion having a first axis and a proximal portion having a second axis oriented approximately perpendicular to the first axis to define a first right angled cannula, which is adapted to be inserted into the superior vena cava of the beating heart to receive blood in the superior vena cava positioned to enter the right atrium of the beating heart; and a second fluid conduit including, for example, a cannula having a distal portion having a first axis and a proximal portion having a second axis oriented approximately perpendicular to the first axis to define a second right angled cannula, which is adapted to be inserted into the inferior vena cava of the beating heart to receive blood in the inferior vena cava positioned to enter the right atrium of the beating heart. According to an alternative embodiment of the present invention, the first and second fluid conduits can include other components adapted for insertion into the respective vena cavae which have various other shapes/configurations as known to the skilled in the art. The apparatus can also include at least one pair of constricting straps or balloons adapted to be positioned to occlude blood flow around outer surface portions of such first and second fluid conduits/vena cavae cannulae when operatively inserted therein, to thereby prevent blood that is inbound to the right atrium from bypassing the first and second fluid conduits/vena cavae cannulae, respectively.

The apparatus can further include a third fluid conduit including, for example, an elongate, right angled, or other shaped, e.g., cannula, adapted to be inserted into the pulmonary artery of the beating heart to convey into the pulmonary artery substantially all blood collected from the superior vena cava and the inferior vena cava. The apparatus can also include a multiport connector including a pair of inlets for individually (separately) receiving blood from each of the first and second conduits/vena cava cannulae, and an outlet for outputting the received blood, and a common manifold extending therebetween to define a Y-connector to thereby connect both the first and second conduits/vena cavae cannula to the inlet side of the pump, along with various lengths of connective tubing.

The apparatus can also include an, e.g., manually operated pump having at least one inlet collectively in fluid communication with both the proximal end portion of the first fluid conduit and the proximal end portion of the second liquid fluid conduit to receive blood from both the first and the second fluid conduits, being substantially all blood flow from each of the superior vena cava and the inferior vena cava respectively; an outlet positioned to convey the substantially all blood flow received from both the superior vena cava and the inferior vena cava into a third fluid conduit to provide substantially complete blood flow to the lungs of a patient undergoing a cardiac procedure; and a flexible body extending between the at least one inlet and the outlet and including an internal cavity having a preselected volume to define a blood reservoir to thereby convey into the third fluid conduit substantially all blood collected from each of the superior vena cava and the inferior vena cava to provide substantially complete blood flow to the lungs of a patient undergoing a cardiac procedure.

Accordingly, each component of the apparatus can be selected to have a volume capacity tailored to a specific body characteristic of the individual patient, e.g., size, height, weight, and/or surface area, etc., based, for example, according to a nomogram indicating, e.g., the expected stroke capacity of the right ventricle of the patient's heart or other related characteristic, to provide a sufficient flow capacity to thereby provide substantially unobstructed blood flow to the pulmonary artery of the beating heart (and thus, to the lungs of the patient) from the vena cavae, when the patient is undergoing the cardiac procedure. That is, when implemented to operatively bypass portions of the right side of a beating heart, each cannula and the reservoir can be selected to provide sufficient capacity to function as described.

In the preferred configuration, the pump is, for example, a hand-operated pump having a main body such as in the form of a bulb syringe, which can act as a blood reservoir. When implemented on a patient, e.g., when each of the cannulas are operatively inserted, the apparatus can form a closed extracardiac pathway from the vena cavae to the pulmonary artery of the beating heart. Advantageously, when blood flow is sufficient, the apparatus can allow a continuous and unassisted flow of blood between the vena cavae and pulmonary artery (and thus, the lungs) through the pump so that, for example, when implemented to bypass portions of the right side of the heart with the left side of the heart beating normally, at least to some substantial extent, portions of the blood pressure to the lungs is provided by the left side of the heart. When blood flow is insufficient, the bulb syringe can be compressed manually to manually assist the pumping of blood into the lungs. Further, the pump can include a checkvalve to prevent a backflow of blood from the pulmonary artery. Still further, the inlet and the outlet to the bulb syringe can be sized so that synergistically, when the bulb syringe is compressed, the blood in the syringe more readily passes through the outlet than through the inlet. This can be accomplished, for example, by having an inlet smaller than the outlet.

Embodiments of the present invention also include a kit containing an apparatus to provide a substantially bloodless field to assist in cardiac procedures. According to an embodiment of the kit, the kit includes a container; and positioned in the container: a plurality of first fluid conduits (e.g., cannulae) having a distal end portion adapted to be inserted into the superior vena cava of a patient, a plurality of second fluid conduits (e.g., cannulae) having a distal end portion adapted to be inserted into the inferior vena cava of a patient, and a plurality of manually operated pumps each comprising a pliable bulb syringe having an inlet, an outlet, and a body including a cavity adapted to provide a blood reservoir for a portion of blood flow received from a selected pair of first and second cannulae when operatively positioned within the patient. The kit also contains at least one multi-port connector having at least three conduit ports and a manifold extending therebetween defining a Y connector adapted to be connected between the selected pair of first and second cannulae and an inlet of a selected one of the plurality of manually operated pumps. The kit also contains at least one, but preferably a plurality of third fluid conduits (e.g., cannulae) adapted to be operatively connected to the outlet of the associated one of the plurality of manually operated pumps to be inserted into the pulmonary artery of the beating heart to form a conduit to thereby convey into the pulmonary artery of the patient substantially all blood collected from the superior and inferior vena cava by the selected pair of first and second cannulae. The plurality of, e.g., first cannulae, the plurality of, e.g., second cannulae, the one or more, e.g., third cannulae, and the volume or reservoir capacity of the bulb syringe, can each be separately “sized” so that specific combinations of individual pairs of first and second cannula, the third cannula, and the manually operated pump fit a different one of a plurality of standard patient body weight sizes of a potential patient to thereby accommodate a substantial majority of potential patients.

Embodiments of the present invention also provide methods of assisting procedures involving the right side of the heart of a patient. For example, a method can include inserting at least portions of a first fluid conduit into the superior vena cava of the beating heart to thereby receive substantially all blood in the superior vena cava positioned to enter the right atrium of the beating heart; inserting at least portions of a second fluid conduit into the inferior vena cava of the beating heart to thereby receive substantially all blood in the inferior vena cava positioned to enter the right atrium of the beating heart; and inserting at least portions of a third fluid conduit into the pulmonary artery of the beating heart, to receive and deliver to the pulmonary artery substantially all blood flow collected from the superior and inferior vena cavae. That is, the first fluid conduit, the second fluid conduit, and the third fluid conduit in combination can form at least portions of an extracardiac pathway to convey into the pulmonary artery the substantially all blood collected from the superior vena cava and the substantially all blood collected from the inferior vena cava to thereby provide a substantially complete and preferably unassisted blood flow from the vena cavae to the lungs of the patient undergoing a cardiac procedure. The method can also include pumping blood, e.g., manually, through the extracardiac pathway when blood flow to the lungs of a patient undergoing a cardiac procedure is insufficient to thereby control the blood flow to the lungs of the patient.

Advantageously, the pumping can involve use of a manually operated bulb syringe with a unidirectional valve at the outflow end to pump blood manually to the patient's lungs. The pump can be inserted in the inferior and superior vena cavae via, e.g., cannulae, or other conduits known to those skilled in the art, to obtain inflow. These cannulae can be either directly or indirectly connected to the pump with a multiport (e.g., Y) connector. The outflow conduit, e.g. an elongate or angled cannula, can be inserted in the pulmonary trunk, with the bulb syringe acting as a reservoir of, e.g., the stroke volume of the right ventricle. The volume of the syringe and the “sizes” of the cannulae can be selected according to the normogram, depending on the body surface area of the patient, or other factors known to those skilled in the art. Whenever blood flow to the lungs is insufficient, the syringe can be compressed manually to pump blood into the pulmonary artery, and thus, into the lungs. The apparatus employed by an embodiment of this method is therefore capable of maintaining cardiac output while the surgeon is working on, for example, the right side of the heart. Advantageously, the patient's lung(s) can be utilized for blood oxygenation during heart surgery, thereby avoiding the need for artificial cardiopulmonary bypass (CPB) circuits and the associated disadvantages of such CPBs.

Advantageously, the methods and apparatus made according to embodiments of the present invention, are extraordinarily simple to employ and can be readily used, for example, in procedures for Tricuspid valve, Atrial septum, Right Atrial tumors excision and Off Pump Coronary Artery Bypass Surgery, although others are within the scope of the present invention.

### BRIEF DESCRIPTION OF THE DRAWINGS

So that the manner in which the features and advantages of the invention, as well as others which will become apparent, may be understood in more detail, a more particular description of the invention briefly summarized above may be had by reference to the embodiments thereof which are illustrated in the appended drawings, which form a part of this specification. It is to be noted, however, that the drawings illustrate only various embodiments of the invention and are therefore not to be considered limiting of the invention's scope as it may include other effective embodiments as well.

FIG. 1 is a partially perspective view of a portion of the circulatory system of a patient primarily illustrating the main components of the heart;

FIG. 2 is a perspective view of an apparatus for assisting in a cardiac procedure according to an embodiment of the present invention;

FIG. 3 is a perspective view of a manually operated pump of the apparatus of FIG. 2 according to an embodiment of the present invention;

FIGS. 4A and 4B are a perspective view of a pair of right angled (substantially L-shaped) vena cavae cannula of the apparatus of FIG. 2 according to an embodiment of the present invention;

FIG. 4C is a perspective view of an elongate pulmonary artery cannula of the apparatus of FIG. 2 according to an embodiment of the present invention;

FIG. 4D is a perspective view of a multiport (wye or “Y”) connector of the apparatus of FIG. 2 according to an embodiment of the present invention;

FIG. 4E is a perspective view of a pair of constrictors used to occlude blood flow surrounding outer surfaces of the vena cavae cannula of FIGS. 4A and 4B, respectively, according to an embodiment of the present invention;

FIG. 5 is a partial environmental perspective view of the apparatus of FIG. 2 according to an embodiment of the present invention;

FIG. 6 is a partial environmental perspective view of an apparatus for assisting in a cardiac procedure utilizing a right angled pulmonary artery cannula in place of the elongate cannula illustrated in FIG. 4C according to an embodiment of the present invention;

FIG. 7 is a partial environmental perspective view of a portion of the apparatus of FIG. 2 illustrating the formation of a substantially bloodless field in the right atrium of the heart according to an embodiment of the present invention; and

FIG. 8 is a perspective view of a kit providing the components of the apparatus for assisting in a cardiac procedure according to an embodiment of the present invention.

### DETAILED DESCRIPTION

The present invention will now be described more fully hereinafter with reference to the accompanying drawings, which illustrate embodiments of the invention. This invention may, however, be embodied in many different forms and should not be construed as limited to the illustrated embodiments set forth herein. Rather, these embodiments are provided so that this disclosure will be thorough and complete, and will fully convey the scope of the invention to those skilled in the art.

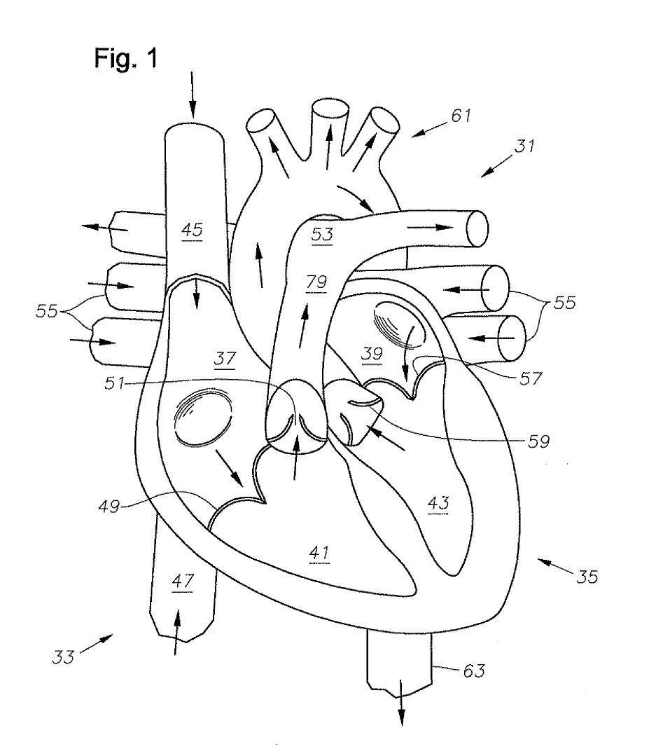

As shown in FIG. 1, the heart 31 is actually composed of two separate pumps 33, 35, lying side-by-side with each other, although the individual pumps 33, 35, are generally referred to as the right side 33 or the left side 35 of the heart 31. Each pump 33, 35, has an upper chamber (atrium) 37, 39, which receives blood and a lower chamber (ventricle) 41, 43, which pumps the blood. The right side 33 of the heart 31 primarily functions to receive deoxygenated blood and to pump the deoxygenated blood to the lungs for oxygenation. The left side 35 of the heart 31 primarily functions to receive the oxygenated blood from the lungs and to pump the oxygenated blood to the body. Specifically, deoxygenated blood destined for the right side 33 of the heart 31 enters the right atrium 37 from superior and inferior vena cavae 45, 47. The right atrium 37 collects the deoxygenated blood from the vena cavae 45, 47 (and the coronary sinus) and then forces the blood through the tricuspid (right atrioventricular) valve 49 and into the right ventricle 41. The right ventricle 41 contracts to force the deoxygenated blood through the pulmonary (semilunar) valve 51 into the pulmonary artery 53, which conveys the deoxygenated blood to both of the lungs, where the blood receives oxygen. The oxygenated blood cycles back from the lungs through the pulmonary veins 55 and to the left atrium 39, which collects the oxygenated blood and then forces the blood through the mitral (left atrioventricular) valve 57 and into the left ventricle 43. The left ventricle 43 contracts to force the oxygenated blood through the aortic (semilunar) valve 59 and into the ascending and descending aorta 61, 63, which conveys the oxygenated blood to the arteries of the body of the patient and eventually back through the veins of the body which collectively return the blood to the heart, under pressure, via either the superior or inferior vena cava 45, 47.

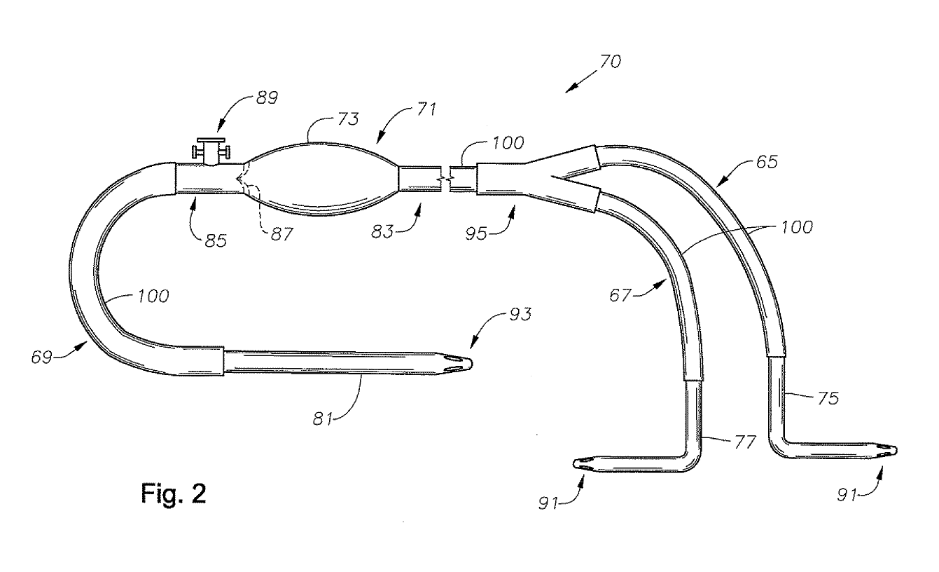

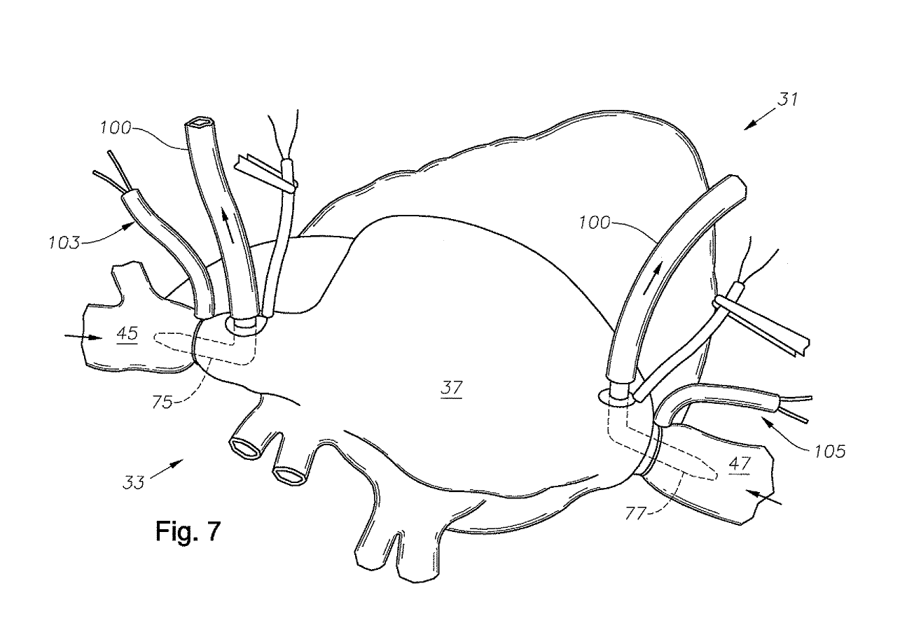

As noted previously, various surgical procedures may be necessary on the heart in order to repair damage or to prevent impending failure, which have in the past required a complete cardio-pulmonary bypass (CPB), along with a complete cessation of all cardiopulmonary activity. FIGS. 2-7 illustrate an apparatus 70 configured for use in performing various procedures including, for example, procedures for the Tricuspid valve, Atrial septum, Right Atrial tumors excision and Off Pump Coronary Artery Bypass Surgery, etc., performed without a complete cessation of all cardiopulmonary activity (typically collectively referred to as beating heart surgery), which allows the surgical team to safely maintain a good cardiac output during such surgery.

According to a generalized version of an embodiment of the apparatus 70, and as will be described in more detail below, apparatus 70 can be utilized to provide an extracardiac pathway between the vena cavae 45, 47, and the pulmonary artery 53. To do so, apparatus 70 can include a manually operated pump 71 including, for example, a bulb syringe 73. The apparatus 70 can be inserted into the inferior and superior vena cavae 45, 47, via a pair of conduits 65, 67, including, e.g., cannula 75, 77, to obtain inflow, and can be inserted into the pulmonary trunk 79 of the pulmonary artery 53 via a conduit 69 including, e.g., cannula 81, 81′, to convey blood flow from the vena cavae 45, 47, and to the lungs for oxygenation. Beneficially, the bulb syringe 73 can act as a reservoir of, e.g., the stroke volume of the right ventricle 41. The volume of the bulb syringe 73 and the sizes of the cannulae 75, 77, 81, 81′, can be selected, for example, according to the normogram, depending upon a specific body characteristic of the individual patient, e.g., size, height, weight, and/or surface area, etc. whenever flow is insufficient, a surgical assistant, for example, can manually compress the bulb syringe 73 to pump blood into the lungs. The apparatus 70 is therefore capable of maintaining cardiac output while the surgeon is working, for example, on the right side 33 of the heart 31. Beneficially, the patient's lung(s) can be utilized for blood oxygenation during heart surgery, and for surgery on the right side 33 of the heart 31, the left side 35 of the heart 31 can be the prime mover of blood flow, thereby avoiding the need for artificial cardiopulmonary bypass (CPB) circuits and the attendant disadvantages of CPB.

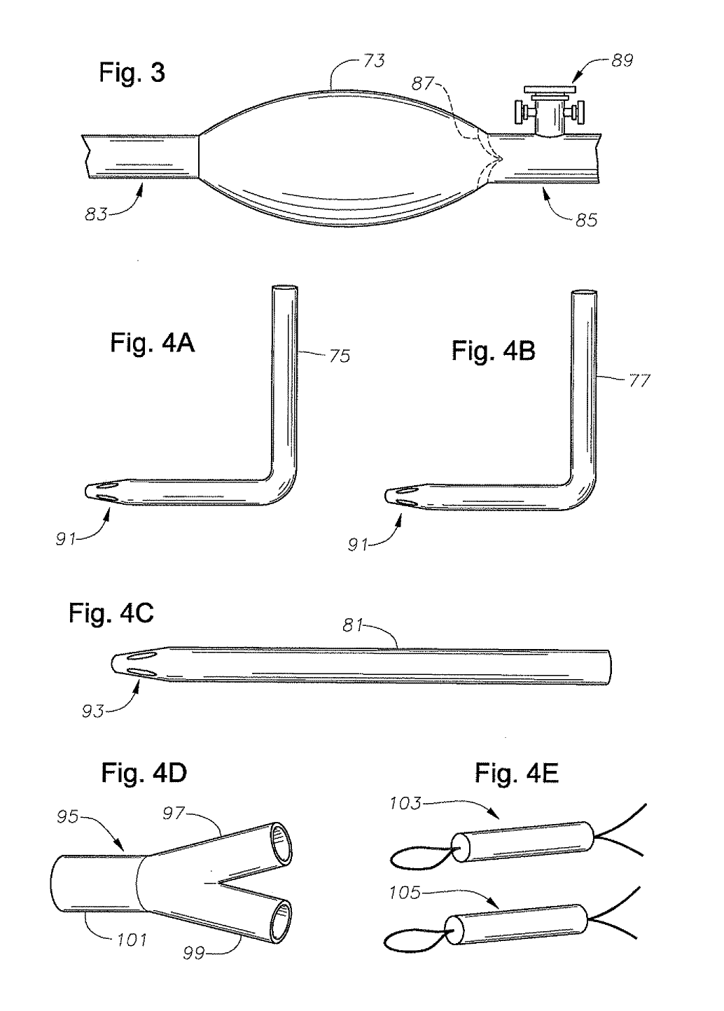

More specifically, as perhaps best shown in FIGS. 2 and 3, according to an embodiment of the present invention, the apparatus 70 can include a manually operated pump 71 including, for example, a pliable-compressible bulb syringe 73 adapted to be manually compressed to form and control a pumping action, to thereby pump blood manually to the patient's lungs. The bulb syringe 73 can include an inlet 83 to receive blood from the vena cavae 45, 47, and an outlet 85 to convey blood to the pulmonary artery 53. According to a preferred configuration, the outlet 85 as a larger diameter than the inlet 83 so that the outlet 85 (and the pulmonary conduit or cannula 81) provides the path of least resistance during compression of the bulb syringe 73. The bulb syringe 73 can include a unidirectional valve 87 located adjacent the outflow end 85 and positioned to prevent a backflow of blood from the pulmonary artery to the syringe 73 during decompression of the bulb syringe 73, for example, after the assistant surgeon releases compression of the bulb syringe 73. The inner surface portions of the bulb syringe 73 can also include an anticoagulate coating (not shown), such as, for example, heparin, or others known to those skilled in the art, to reduce the potential for forming blood clots. The manually operated pump 71can also include a connector, e.g., three-way connector 89, positioned adjacent the outlet 85 of the bulb syringe 73 to receive medications and fluid and to transmit the medications or fluid into the pulmonary artery 53.

As perhaps best shown in FIGS. 2 and 4A-4B, the apparatus 70 can include a first conduit including, e.g., cannula 75, adapted to be inserted into the superior vena cava 45 of a beating heart 31 to receive blood in the superior vena cava 45 prior to entry into the right atrium 37 of the beating heart; and a second conduit including, e.g., cannula 77, adapted to be inserted into the inferior vena cava 47 of the beating heart 31 to receive blood in the inferior vena cava 47 prior to entry into the left atrium 39 of the beating heart 31. Although the cannula 75, 77, can have any angular deflection as known to those skilled in the art, according to a preferred configuration, both cannula 75, 77, are in the form of right-angled or L-shaped cannula 75, 77, as illustrated in FIGS. 4A-4B, to enhance insertion into the vena cavae 45, 47, respectively, and to reduce any lateral torque action once inserted within the respective vena carvae 45, 47. Further, the cannula 75, 77, can each include at least one but preferably a plurality of inlets 91 positioned to receive blood therethrough.

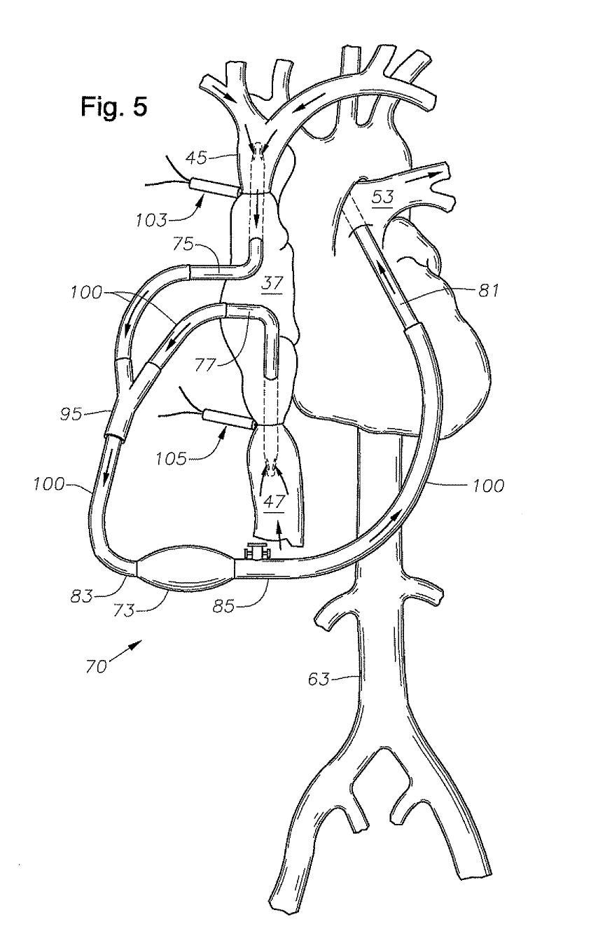

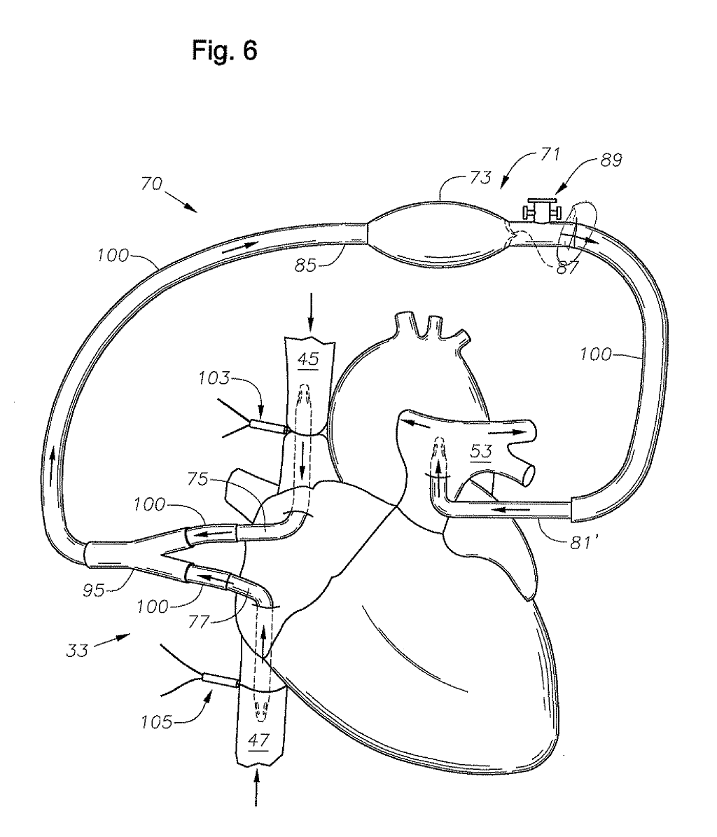

As perhaps best shown in FIGS. 2 and 4C, the apparatus 70 can include a third conduit including, e.g., cannula 81, adapted to be inserted into the pulmonary artery of the beating heart to convey into the pulmonary artery 53 the blood collected from the superior and the inferior vena cavae 45, 47. Although the cannula 81 can have any angular deflection as known to those skilled in the art, according to a preferred configuration, the cannula 81 is in the form of an elongate (“straight”) cannula 81 (FIGS. 4C and 5) or an angled (e.g., “right-angled” or “L-shaped”) cannula 81′ (FIG. 6) to enhance insertion into the pulmonary artery 53 and/or to reduce any lateral torque action once inserted within the pulmonary artery 53. The cannula 81, 81′, can include at least one, but preferably a plurality of inlets 93 positioned to expel blood therethrough.

As perhaps best shown in FIGS. 2 and 4D, the apparatus 70 can include a connector, e.g., Y-connector 95, having legs 97, 99, adapted to receive a proximal end of each of the cannula 75, 77, and leg 101 adapted to interface with input 83 of the bulb syringe 73. As with other portions of the apparatus 70, various lengths of flexible connector tubing 100, as known to those skilled in the art, can be used as an interface between the various components of the apparatus 70. Such tubing 100 can have various lengths and volumetric capacities between the various components of the apparatus 70 to complete the first, second, and third fluid conduits 65, 67, 69. Alternatively, at least segments of such tubing can be integral with the inlet 83 and/or outlet 85 of the manually operated pump 71. In such configuration, the integral portions of the connection tubing can be made of the same material as the bulb syringe 73, depending upon the manufacturing process used to form the manually operated pump 71.

As shown in FIG. 4E, apparatus 70 can also include a pair of constrictors 103, 105, as known and understood by those skilled in the art, which can be, for example, inserted around an outer wall of each respective vena cavae 45, 47 (see, e.g. FIGS. 5-7) to compressively hold the inner walls of the vena cava 45, 47, in sealing contact with an outer surface portion of the respective cannula 75, 77, to thereby restrict blood from flow in around the outside of the cannula 75, 77, and into the right atrium 37. The constrictors 103, 105, can include surgical silicone, which can be, e.g., twist-tied, etc., to form a tourniquet-like seal. Alternatively, each respective cannula 75, 77, can carry an external balloon (not shown) or other occlusion means as known to those skilled in the art, which can similarly function to occlude any potential blood pathway between the inner walls of the respective vena cava 45, 47, and the external services of the cannula 75, 77, when inserted.

Beneficially, as perhaps best shown in FIG. 5, the combination of the vena cavae cannula 75, 77, the Y-connector 95, the pump 71, and the pulmonary artery cannula 81, 81′, when assembled and when inserted into the patient, and when secured using, for example, constrictors 103, 105, form a closed extracardiac pathway from the vena cavae 45, 47, to the pulmonary artery 53 of the heart 31. Note, each portion or section of the extracardiac pathway can provide a sufficient flow capacity so that the apparatus 70 when operatively inserted into the patient can provide the substantially complete blood flow to the lungs of the patient undergoing a cardiac procedure, to thereby allow the surgical team to substantially operatively bypass, for example, the right side 33 of the beating heart 31. As noted above, to ensure sufficient flow capacity, the volume of the bulb syringe 73 and the sizes (flow capacity) of the cannulae 75, 77, 81, can be selected, for example according to the normogram, depending upon a specific body characteristic of the individual patient, e.g., size, height, weight, and/or surface area, etc. Whenever flow is insufficient, a surgical assistant, for example, can manually compress the bulb syringe 73 to increase the blood flow to the lungs. Similarly, decompression of the bulb syringe 73 can cause a slight vacuum to enhance blood flow from the vena cavae 45, 47. As noted previously, valve 87 can function to prevent back flow of blood from the pulmonary artery 53 during such decompression.

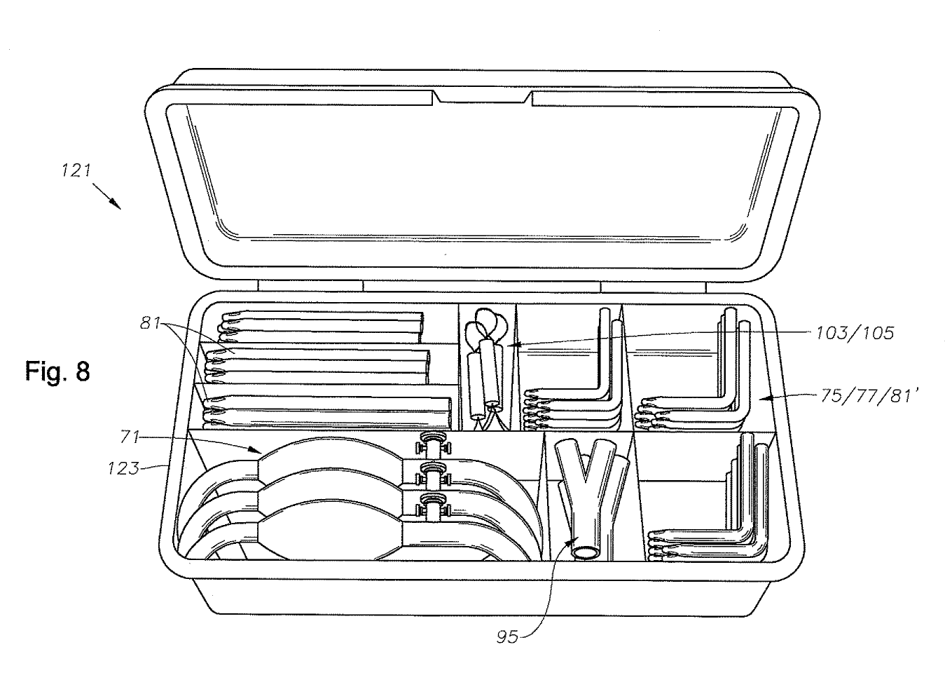

Beneficially, embodiments of the present invention provide a kit 121 containing components of the apparatus 70 to include a plurality of different size/capacity vena cavae cannulac 75, 77, one or more Y connectors 95, a plurality of pumps 71 having various, e.g., standard volume bulb syringes 73, a plurality of different size/capacity pulmonary artery cannulae 81, 81′, and/or at least a pair of constrictors 103, 105. Specifically, as perhaps best shown in FIG. 8, according to an embodiment of the kit 121, the kit 121 can include a container 123; and positioned in the container 123: a plurality of first cannulae 75 adapted to be inserted into the superior vena cava 45 of a patient, a plurality of second cannulae 77 adapted to be inserted into the inferior vena cava 47 of a patient, and a plurality of manually operated pumps 71 each comprising a pliable bulb syringe 73 having an inlet 83, an outlet 85, and a body including a cavity adapted to provide a blood reservoir for blood received from a different pair of first and second cannulae 75, 77, when operatively positioned within the patient

The kit 121 can also contain at least one Y connector 95 adapted to be connected between a selected pair of first and second cannulae 75, 77, and an inlet 83 of an associated one of the plurality of manually operated pumps 71, and at least one, but preferably a plurality of third cannula 81, 81′, adapted to be operatively connected to the outlet 85 of the associated one of the plurality of manually operated pumps 71, and adapted to be inserted into the pulmonary artery 53 of the beating heart to form a conduit to convey into the pulmonary artery 53 of the patient substantially all blood collected from the superior and inferior vena cava 45, 47, by the selected pair of first and second cannulae 75, 77.

Beneficially, the plurality of first cannulae 75, the plurality of second cannulae 77, the plurality of third cannulae 81, 81′, and the volume of the bulb syringe 73, can be each separately sized so that specific combinations of individual pairs of first and second cannulae 75, 77, the third cannula 81, 81′, and the manually operated pump 71, fit a different one of a plurality of standard patient body weight sizes of a potential patient to thereby accommodate a substantial majority of potential patients. Also beneficially, either one or both of the selected first and second vena cavae cannulae 75, 77, can be in the form of an angled (e.g., right-angled) cannula. Further, although the pulmonary artery cannula 81, 81′, is in the form of an elongated (straight) cannula 81, according to a preferred configuration, the third pulmonary artery cannula can instead be an angled (e.g., right angled) cannula 81′ or other shape as necessary to reduce torque within the pulmonary artery resulting from the positioning of the cannula 81, 81′.

Inner surface portions of the bulb syringe 73 of the selected manually operated pump 71 can include a heparin coating (not shown) or other anticoagulant to limit the potential for blood clotting resulting in the formation of dangerous blood clots. The selected manually operated pump 71 can also include a valve 87 positioned between the inlet 83 and the outlet 85 of the selected manually operated pump 71 to prevent a backflow of blood from the pulmonary artery 53 to the syringe 73, when the third cannula 81, 81′, is operatively positioned in the pulmonary artery 53.

Further, the kit can include a pair of constrictors 103, 105, positioned in the container 121, with at least one adapted to be tightened around a portion of the superior vena cava 45 to thereby form a seal between an inner wall of the superior vena cava 45 and an outer surface of the selected first cannula 75, and the other adapted to be tightened around a portion of the inferior vena cava 47 to thereby form a seal between an inner wall of the inferior vena cava 47 and an outer surface of the second cannula 77. The kit can also include various lengths of flexible connector tubing 100 (not shown in FIG. 8). Such tubing 100, however, is generally readily available and need not be included in the kit.

As perhaps best shown in FIGS. 5-7, embodiments of the present invention can also include methods of providing a bloodless field to assist in procedures involving the right side of the heart of a patient. Please note, although other forms of blood and fluid conduits can be used, e.g. catheters, etc., for simplicity, the following embodiments will be described with respect to cannula only, and with reference to apparatus 70. Such a method of providing a bloodless field to assist in procedures involving the right side of the heart of a patient can include first selecting appropriately sized components to assemble apparatus 70. That is, a member of the surgical team or other personnel can obtain, e.g., a normogram to allow, for example, a surgical team member to determine the appropriate volume for the bulb syringe 73; determine the appropriate size/capacity of the vena cavae cannulae 75, 77, sized for the respective vena cavae 45, 47, for example, and determine the appropriate size/capacity of the pulmonary artery cannula 81, 81′. The appropriate volume of the bulb syringe 73 and the appropriate size/capacity of the vena cava cannulae 75, 77 and pulmonary artery cannula 81, 81′, for example, can be based upon a specific body characteristic of the individual patient, e.g., size, height, weight, and/or body surface area, etc., of the patient.

The method can also include connecting the components together to “build” a customized form of apparatus 70 customized to the specific patient. That is, if the various components of apparatus 70 are not already connected together as described above, the method can include, in no particular order, assembling the components by connecting the first and the second vena cava cannulae 75, 77, to the respective legs 97, 99, of the Y connector 95; connecting leg 101 of the Y connector 95 to the inlet portion 83 of the, e.g., bulb syringe 73 of the pump 71, either directly or via an additional fluid conduit, e.g., tubing 100; and connecting the pulmonary artery cannula 81 to the outlet portion 85 of the, e.g., bulb syringe 73 of the pump 71, again, either directly or via an additional fluid conduit, e.g., tubing 100.

The method also includes, e.g., cannulating the superior and inferior vena cavae 45, 47, using the respective vena cava cannula 75, 77, either directly or through respective incisions in the right atrium 37, and cannulating the pulmonary artery 53 with the pulmonary artery cannula 81, 81′, for example, at the trunk 79. FIG. 5 illustrates an elongate (straight) pulmonary artery cannula 81. FIG. 6 illustrates use of a right-angle pulmonary artery cannula 81′. Note, various clamps as known to those skilled in the art can be used to prevent any undesirable flow of blood through the cannula 81, 81′, during the cannulating process. Note also, various clamps and/or sutures, etc., as known to those skilled in the art can be used to prevent any undesirable flow of blood around the entry incision of the cannula 75, 77, 81, 81′.

Once inserted, the blood pathway between the outer surface of the respective vena cava cannulae 75, 77, and the inner walls of the vena cavae 45, 47, respectively, can be sealed using, for example, constrictors 103, 105, or others as known and understood by those skilled in the art, which can be, for example, inserted around an outer wall of each respective vena cavae 45, 47 (see, e.g. FIGS. 5-7) to compressively hold the inner walls of the vena cava 45, 47, in sealing contact with an outer surface portion of the respective cannula 75, 77, to thereby restrict blood from flow around the outside of the cannula 75, 77, and into the right atrium 37.

Once completed, as perhaps best shown in FIGS. 5 and 6, the combination of the vena cavae cannula 75, 77, the Y-connector 95, the pump 71, and the pulmonary artery cannula 81, 81′, form a closed extracardiac pathway from the vena cavae 45, 47, to the pulmonary artery 53 of the heart 31, to thereby provide substantially complete blood flow to the lungs of the patient undergoing the cardiac procedure. In this illustrative example, such components substantially operatively bypass the right side 33 of a beating heart 31, to form a substantially bloodless field to aid the surgeon in performing the cardiac procedure on the right side 33 of the beating heart 31.

Beneficially, when using properly sized components, the blood pressure provided by the left side 35 of the beating heart 31 may be sufficient to circulate the blood to the lungs of the patient. Whenever flow is insufficient, however, a surgical assistant, for example, can iteratively manually compress the bulb syringe 73, pumping blood through the extracardiac pathway, to increase the blood flow to the lungs, i.e., control blood flow in the pulmonary artery cannula 81, 81′, to thereby control the blood flow to the lungs of the patient. Note, a bi-leaf valve 87, for example, within the bulb syringe 73 or outlet section 85 can function to prevent such backflow of blood from the pulmonary artery cannula 81, 81′, and back into the bulb syringe 73 during decompression of the bulb syringe 73. Note, the method can further include inserting medications and fluid into the extracardiac pathway via an, e.g., three-way connector 89, to transmit the medications or fluid into the pulmonary artery 53.

In the drawings and specification, there have been disclosed typical preferred embodiments of the invention, and although specific terms are employed, the terms are used in a descriptive sense only and not for purposes of limitation. The invention has been described in considerable detail with specific reference to these illustrated embodiments. It will be apparent, however, that various modifications and changes can be made within the spirit and scope of the invention as described in the foregoing specification. For example, although the description primarily focuses on application to the right atrium of the heart, with proper selection of the input cannula or other conduits and the output cannula or other conduits, the apparatus 70 can be utilized as a manual blood bypass on other portions of the body of either human or animal. Further, although the manually operated pump was described as incorporating a hand-operated bulb syringe, the bulb syringe could be replaced with a similar electric pump such as, for example, one utilizing a flexible diaphragm, which could be manually controlled or monitored by, for example, a surgical team member.

1. An apparatus to provide a substantially bloodless field to assist in cardiac procedures involving the right side of a beating heart of a patient, the apparatus comprising:
a first liquid fluid conduit including a proximal end portion, a medial portion, and a distal end portion adapted to be inserted into the superior vena cava of the beating heart to continuously receive substantially all blood flow in the superior vena cava to enter the right atrium of the beating heart;
a first constrictor adapted to be tightened around a portion of the superior vena cava to thereby form a seal between an inner wall of the superior vena cava and an outer surface of the distal end portion of the first fluid conduit to thereby substantially prevent blood located upstream of the distal end portion of the first liquid fluid conduit from entering the right atrium of the beating heart from the superior vena cava when the distal end portion of the first liquid fluid conduit is inserted into the superior vena cava;
a second liquid fluid conduit including a proximal end portion, a medial portion, and a distal end portion adapted to be inserted into the inferior vena cava of the beating heart to continuously receive substantially all blood flow in the inferior vena cava to enter the right atrium of the beating heart;
a second constrictor adapted to be tightened around a portion of the inferior vena cava to thereby form a seal between an inner wall of the inferior vena cava and an outer surface of the distal end portion of the second fluid conduit to thereby substantially prevent blood located upstream of the distal end portion of the second liquid fluid conduit from entering the right atrium of the beating heart from the inferior vena cava when the distal end portion of the second liquid fluid conduit is inserted into the inferior vena cava;
a manually operated pump positioned to receive blood therein and to manually pump blood therefrom when blood is contained within the manually operated pump, the manually operated pump comprising at least one inlet connected to and collectively in liquid fluid communication with both the proximal end portion of the first fluid conduit and the proximal end portion of the second liquid fluid conduit to thereby receive blood flowing from both the first and second fluid conduits, the blood flowing from the first and second fluid conduits being substantially all blood flowing from each of the superior vena cava and the inferior vena cava respectively, the manually operated pump also comprising an outlet positioned to convey the substantially all blood received from both the superior vena cava and the inferior vena cava, and a flexible body extending between the at least one inlet and the outlet and including an internal cavity having a preselected volume to define a blood reservoir; and
a third liquid fluid conduit having a proximal end portion connected to and in fluid communication with the outlet of the manually operated pump to receive the substantially all blood received by the manually operated pump from the superior vena cava and the inferior vena cava, and having a distal end portion adapted to be inserted into the pulmonary artery of the beating heart of the patient to convey into the pulmonary artery the substantially all blood received by the manually operated pump from both the superior vena cava and the inferior vena cava, the manually operated pump and the first, second, and third conduits when operatively inserted into the patient in combination form a substantially mechanically unobstructed closed extracardiac pathway to the pulmonary artery of the beating heart from the vena cavae, the pathway having a sufficient flow capacity to operatively provide for conveying the substantially all blood received from the superior vena cava and the inferior vena cava into the pulmonary artery to thereby supply deoxygenated blood to the lungs of the patient for oxygenation when undergoing the cardiac procedure, substantially operatively bypassing the right side of the beating heart.

2. An apparatus as defined in claim 1,
wherein the at least one inlet of the manually operated to pump comprises a single inlet adapted to receive the blood flowing from both the first and the second conduits to define a consolidated blood receiving inlet;
wherein the manually operated pump further comprises a pliable bulb syringe positioned between and connected to the inlet and the outlet of the pump to convey blood between the inlet and the outlet of the pump when the distal end portion of the first conduit, the distal end portion of the second conduit, and the distal end portion of the third conduit are operatively inserted into the patient to form the closed extracardiac pathway and positioned to provide manual user assist to the blood flowing into the third fluid conduit when blood flow to the lungs is insufficient to thereby provide control of the blood flow to the lungs of the patient; and
wherein the apparatus further comprises a connector having three conduit ports and a common manifold extending between the three conduit ports to define a Y-connector, the first conduit port connecting to the proximal end portion of the first conduit to receive the blood flowing therefrom, the second conduit port connecting to the proximal end portion of the second conduit to receive the blood flowing therefrom, and the third conduit port in fluid communication with the consolidated blood receiving inlet of the manually operated pump to convey to the manually operated pump the blood received from both the first and second conduits by the Y-connector.

3. An apparatus as defined in claim 2,
wherein the distal end portion of first fluid conduit comprises at least a portion of a first cannula;
wherein the distal end portion of the second fluid conduit comprises at least a portion of a second cannula;
wherein the distal end portion of third fluid conduit comprises at least a portion of a third cannula defining a pulmonary artery cannula;
wherein the manually operated pump includes a one-way valve positioned adjacent the outlet of the pump and positioned in fluid communication with the third conduit to prevent a backflow of blood from the pulmonary artery into the bulb syringe; and
wherein the reservoir of the bulb syringe has a volume selected according to a specific body characteristic of the patient to thereby provide a reservoir capacity that is approximately equivalent to a stroke volume of the right ventricle of the right side of the heart of the patient.

4. An apparatus as defined in claim 3,
wherein an inner wall of the pliable bulb syringe includes a heparin coating positioned thereon to limit the potential for blood clotting resulting in the formation of dangerous blood clots; and
wherein the pump includes a multi-port connector having at least three inlet ports to thereby define a three-way connector, the three-way connector positioned downstream of the outlet of the manually operated pump and downstream of the one-way valve to receive medications and liquid fluid and to convey the medications and liquid fluid to the pulmonary artery when inserted through at least one of the at least three inlet ports.

5. An apparatus as defined in claim 4, wherein the first cannula includes a first distal portion having a first axis and a second proximal portion having a second axis oriented approximately perpendicular to the first axis of the first cannula to define a first right-angled cannula, wherein the second cannula comprises a first distal portion having a first axis and a second proximal portion having a second axis oriented approximately perpendicular to the first axis of the second cannula to define a second right-angled cannula, and wherein each of the first and second cannulae are sized for the respective vena cavae according to the specific body characteristics of the patient.

6. An apparatus to provide a substantially bloodless field to assist in cardiac procedures involving a heart of a patient, the apparatus comprising:
a first liquid fluid conduit including a proximal end portion and a distal end portion adapted to be inserted into the superior vena cava of the heart to thereby receive blood flow in the superior vena cava positioned to enter the right atrium of the heart;
first occlusion means adapted to occlude blood flow between an inner wall of the superior vena cava and an outer surface of the distal end portion of the first fluid conduit to thereby substantially prevent blood located upstream of the distal end portion of the first fluid conduit from entering the right atrium of the beating heart from the superior vena cava when the distal end portion of the first fluid conduit is operatively inserted into the superior vena cava;
a second liquid fluid conduit including a proximal end portion and a distal end portion adapted to be inserted into the inferior vena cava of the heart to thereby receive blood flow in the inferior vena cava positioned to enter the right atrium of the heart;
second occlusion means adapted to occlude blood flow between an inner wall of the inferior vena cava and an outer surface of the distal end portion of the second fluid conduit to thereby substantially prevent blood located upstream of the distal end portion of the second fluid conduit from entering the right atrium of the beating heart from the inferior vena cava when the distal end portion of the second fluid conduit is operatively inserted into the inferior vena cava;
a pump configured to be positioned to receive blood therein and to manually pump blood therefrom when blood is contained within the pump, the pump comprising at least one inlet adapted to be connected to and collectively in liquid fluid communication with both the proximal end portion of the first fluid conduit and the proximal end portion of the second liquid fluid conduit to thereby receive blood flowing from both the first and second fluid conduits, the blood flowing from the first and second fluid conduits being substantially all blood flowing from each of the superior vena cava and the inferior vena cava respectively, the pump further comprising an outlet positioned to convey the substantially all blood received from both the superior vena cava and the inferior vena cava, and a flexible body extending between the at least one inlet and the outlet and including an internal liquid holding cavity having a volume to define a blood reservoir; and
a third liquid fluid conduit having a proximal end portion adapted to be connected to and in fluid communication with the outlet of the pump to receive the substantially all blood received by the pump from the superior vena cava and the inferior vena cava, and having a distal end portion adapted to be inserted into the pulmonary artery of the beating heart to convey into the pulmonary artery the substantially all blood received by the pump from both the superior vena cava and the inferior vena cava, the pump and the first, second, and third conduits when operatively inserted into the patient in combination form a closed extracardiac pathway from the vena cavae to the pulmonary artery of the beating heart to thereby operatively provide for conveying to the pulmonary artery the substantially all blood received from the superior vena cava and the inferior vena cava to supply deoxygenated blood to the lungs of the patient for oxygenation when undergoing the cardiac procedure, substantially operatively bypassing the right side of the beating heart.

7. An apparatus as defined in claim 6,
wherein the at least one inlet of the pump comprises a single inlet for receiving the blood flowing from both the first and the second conduits to define a consolidated blood flow receiving inlet;
wherein the closed extracardiac pathway is substantially mechanically unobstructed in at least one direction so that the right side of the heart can provide sufficient blood flow to the lungs of the patient when undergoing the cardiac procedure;
wherein the pump comprises a pliable bulb syringe positioned between and connected to the inlet and the outlet of the pump to convey blood flow between the inlet and the outlet of the pump and to provide manual user assist to the blood flowing into the third fluid conduit when blood flow to the lungs of the patient is insufficient to thereby control the blood flow to the lungs of the patient when the distal end portion of the first conduit, the distal end portion of the second conduit, and the distal end portion of the third conduit, are each operatively inserted into the patient to form the closed extracardiac pathway; and
wherein the apparatus further comprises a multi-port connector having three conduit ports and a common manifold extending therebetween to define a Y-connector, the first conduit port connecting to the proximal end portion of the first conduit to receive blood flowing therefrom, the second conduit port connecting to the proximal end portion of the second conduit to receive blood flowing therefrom, and the third conduit port in fluid communication with the consolidated blood receiving inlet of the pump to convey to the blood reservoir of the pump the blood received from the first and second conduits by the Y-connector.

8. An apparatus as defined in claim 7,
wherein the pump includes a one-way valve positioned adjacent the outlet of the pump and in fluid communication with the third conduit to prevent a backflow of blood from the pulmonary artery to the bulb syringe;
wherein the pliable bulb syringe includes an anticoagulant coating positioned therein to limit the potential for blood clotting resulting in formation of dangerous blood clots; and
wherein the reservoir of the bulb syringe has a volume selected according to a specific body characteristic of the patient to thereby provide a reservoir capacity that is approximately equivalent to a stroke volume of the right ventricle of the right side of the heart of the patient.

9. An apparatus as defined in claim 8,
wherein the distal end portion of first fluid conduit comprises at least a portion of a first cannula;
wherein the distal end portion of the second fluid conduit comprises at least a portion of a second cannula;
wherein the distal end portion of third fluid conduit comprises at least a portion of a third cannula defining a pulmonary artery cannula;
wherein the first cannula includes a first distal portion having a first axis and a second proximal portion having a second axis oriented approximately perpendicular to the first axis of the first cannula to define a first right-angled cannula;
wherein the second cannula includes a first distal portion having a first axis and a second proximal portion having a second axis oriented approximately perpendicular to the first axis of the second cannula to define a second right-angled cannula, each of the first and second cannulae being sized for the respective vena cavae according to the specific body characteristics of the patient; and
wherein the third cannula includes a substantially elongate body to define a third straight cannula, the third straight cannula sized according to the specific body characteristics of the patient to have sufficient capacity to convey to a portion of the pulmonary artery the substantially all blood received from the superior and the inferior vena cavae.

10. A kit including components of an apparatus to provide a substantially bloodless field to assist in cardiac procedures, the kit comprising:
a container;
a plurality of first cannulae each positioned in the container and each including a distal end portion being adapted to be inserted into the superior vena cava of the beating heart to receive blood flow in the superior vena cava when positioned to enter the fight atrium of the beating heart, the plurality of first cannulae each separately sized to fit a different one of a plurality of standard patient body sizes of a potential patient to thereby accommodate a substantial majority of potential patients;
a plurality of second cannulae each positioned in the container and each including the distal end portion being adapted to be inserted into the inferior vena cava of the beating heart to receive blood flow in the inferior vena cava when positioned to enter the right atrium of the beating heart, the plurality of second cannulae each separately sized to fit a different one of the plurality of different standard patient body sizes;
a plurality of manually operated pumps each positioned in the container and each comprising a pliable bulb syringe having an inlet, an outlet, and a body, the body including a cavity adapted to provide a blood reservoir for a portion of blood flow received from a selected pair of first and second cannulae when operatively positioned within a patient, the plurality of bulb syringes each separately sized to have a different volume coinciding with a corresponding different one of the plurality of different standard body sizes;
at least one connector positioned in the container and having three conduit ports and a common manifold extending therebetween to define a Y-connector, the first conduit port adapted to be connected to and in fluid communication with a proximal end portion of the first cannula to receive the blood flow therefrom, the second conduit port adapted to be connected to and in fluid communication with a proximal end portion of the second cannula to receive the blood flow therefrom, and the third conduit port adapted to be connected to and in fluid communication with the inlet of a selected one the plurality of manually operated pumps to convey the blood received from the first and the second cannula to the selected manually operated pump; and
at least one third cannula positioned in the container and including a proximal end portion being adapted to be operatively connected to the outlet of the selected manually operated pump, and a distal end portion being adapted to be inserted into the pulmonary artery of the beating heart to form a substantially mechanically unobstructed closed extracardiac pathway in at least one direction to convey to the pulmonary artery substantially all blood collected from the superior and inferior vena cava by the selected pair of first and second cannulae.

11. A kit as defined in claim 10, further comprising:
a first constrictor positioned in the container and adapted to be tightened around a portion of the superior vena cava to thereby form a seal between an inner wall of the superior vena cava and an outer surface of the selected first cannula to thereby substantially prevent blood located upstream of the first cannula from entering the right atrium of the beating heart from the superior vena cava when the distal end portion of the first cannula is operatively positioned within the superior vena cava; and
a second constrictor positioned in the container and adapted to be tightened around a portion of the inferior vena cava to thereby form a seal between an inner wall of the inferior vena cava and an outer surface of the second cannula to thereby substantially prevent blood located upstream of the second cannula from entering the right atrium of the beating heart from the superior vena cava when the distal end portion of the second cannula is operatively positioned within the inferior vena cava.

12. A kit as defined in claim 11,
wherein an inner wall surface of the bulb syringe of the selected manually operated pump includes an anticoagulant coating positioned thereon; and
wherein the selected manually operated pump includes a valve positioned between and in fluid communication with the inlet and the outlet of the selected manually operated pump to prevent a backflow of blood from the pulmonary artery into the bulb syringe when the distal end portion of the third cannula is operatively positioned within the pulmonary artery.

13. A method of providing a substantially bloodless field to assist in procedures involving the right side of the heart of a patient, the method comprising the steps of:
inserting a distal portion of a first fluid conduit into the superior vena cava of the heart of a patient when beating to thereby receive substantially all blood flow in the superior vena cava positioned to enter the right atrium of the beating heart;
inserting a distal portion of second fluid conduit into the inferior vena cava of the beating heart to thereby receive substantially all blood flow in the inferior vena cava positioned to enter the right atrium of the beating heart;
inserting a distal portion of third fluid conduit into the pulmonary artery of the beating heart to convey substantially all blood collected from the vena cavae into the pulmonary artery, the third fluid conduit positioned to receive the substantially all blood collected from the vena cavae, the first, second, and third fluid conduits forming substantial portions of a substantially unobstructed closed extracardiac pathway to convey into the pulmonary artery the substantially all blood collected from the vena cavae; and
manually pumping blood through the extracardiac pathway when blood flow to the lungs of a patient undergoing a cardiac procedure is insufficient to thereby control the blood flow to the lungs of the patient.

14. A method as defined in claim 13, further comprising the steps of:
tightening a first constrictor around a portion of the superior vena cava to thereby form a seal between an inner wall of the superior vena cava and an outer surface of a portion of the inserted distal portion of the first fluid conduit to thereby substantially prevent blood located upstream of the portion of the inserted distal portion of the first fluid conduit from entering the right atrium of the beating heart from the superior vena cava when the distal portion of the first fluid conduit is operatively positioned within the superior vena cava; and
tightening a second constrictor around a portion of the inferior vena cava to thereby form a seal between an inner wall of the inferior vena cava and an outer surface of a portion of the distal portion of the second fluid conduit to thereby substantially prevent blood located upstream of the portion of the inserted distal portion of the second fluid conduit from entering the right atrium of the beating heart from the inferior vena cava when the distal portion of the second fluid conduit is operatively positioned within the superior vena cava.

15. A method as defined in claim 13, wherein the step of manually pumping blood though the extracardiac pathway comprises the steps of:
providing a manually operated pump comprising a collapsible bulb syringe positioned between an inlet and an outlet of the pump to convey blood between the inlet and the outlet and to provide manual user assist to the blood entering in the third fluid conduit when blood flow to the lungs of the patient is insufficient; and
iteratively manually compressing the collapsible bulb syringe to control blood flow in the third fluid conduit when blood flow to the lungs of the patient is insufficient to thereby provide control of the blood flow to the lungs of the patient.

16. A method as defined in claim 13, wherein the first fluid conduit comprises a first cannula, wherein the second fluid conduit comprises a second cannula, wherein the third fluid conduit comprises a third cannula defining a pulmonary artery cannula, wherein the first, second, and third cannulae when operatively positioned in the superior vena cava, inferior vena cava, and pulmonary artery, respectively, form substantial portions of the substantially unobstructed extracardiac pathway to the pulmonary artery of the beating heart from the vena cavae to thereby substantially operatively bypass the right side of the beating heart of the patient when undergoing a cardiac procedure.

17. A method as defined in claim 16, wherein the step of manually pumping blood through the extracardiac pathway comprises the steps of:
providing a pump comprising a pliable but syringe having an internal cavity defining a blood flow reservoir;
operatively connecting the first cannula, the second cannula, and an inlet of the pliable bulb syringe in liquid fluid communication with a first, a second, and a third fluid connection port, respectively, of a multi-port connector defining a Y-connector, the Y-connector having at least three fluid connection ports and an internal manifold extending therebetween; and
operatively connecting the third cannula in liquid fluid communication with an outlet of the bulb syringe so that the bulb syringe is positioned within the extracardiac pathway.

18. A method as defined in claim 17, further comprising the steps of:
selecting the first and second cannulae, each sized for the respective vena cavae according to a specific body characteristic of the patient;
selecting the third cannula sized for the pulmonary artery of the patient according to the specific body characteristic of the patient; and
selecting the bulb syringe sized so that a volume of the blood flow reservoir is approximately equal to an expected stroke volume of the right ventricle of the heart of the specific patient.

19. A method as defined in claim 13, further comprising the steps of:
providing a pump comprising a pliable bulb syringe including an internal cavity defining a blood flow reservoir having a volume preselected according to a specific body characteristic of the patient;
applying an anticoagulant coating within the bulb syringe to thereby reduce blood clotting potential;
providing a one-way valve at least partially within portions of the pump to prevent a backflow of blood from the pulmonary artery to the bulb syringe when the distal portion of the third fluid conduit is operatively positioned in the pulmonary artery;
positioning the bulb syringe within the extracardiac pathway; and
iteratively compressing pliable portions of the bulb syringe to control the blood flow to the lungs of the patient undergoing the cardiac procedure when blood flow circulating through the extracardiac pathway is otherwise insufficient.

20. A method as defined in claim 13, wherein the first cannula includes a first distal portion having a first axis and a second proximal portion having a second axis oriented approximately perpendicular to the first axis of the first cannula to define a first right-angled cannula, and wherein the second cannula includes a first distal portion having a first axis and a second proximal portion having a second axis oriented approximately perpendicular to the first axis of the second cannula to define a second right-angled cannula, the method further comprising the steps of:
sizing the first and second cannulae for the respective vena cavae according to a specific body characteristic of the patient;
operatively positioning the distal portion of the first right angled cannula upstream of an incision in the superior vena cava; and
operatively positioning the distal portion of the second right angled cannula upstream of an incision in the inferior vena cava.

21. A method of providing a substantially bloodless field to assist in procedures involving the right side of the heart of a patient, the method comprising the step of:
maintaining a substantially mechanically unobstructed flow of blood to the lungs of a patient though a substantially closed extracardiac pathway extending to the pulmonary artery of a beating heart of the patient from both the superior vena cava and the inferior vena cava when undergoing a cardiac procedure.

22. A method as defined in claim 21, wherein the step of maintaining a substantially mechanically unobstructed flow of blood to the lungs includes pumping blood through the extracardiac pathway by performing the step of compressing a collapsible bulb syringe positioned within the extracardiac pathway when blood flow to the lungs of the patient undergoing the cardiac procedure is insufficient to thereby control the blood flow to the lungs of the patient.

23. A method as defined in claim 22,
wherein the extracardiac pathway is at least partially provided by a multi-port connector including a common manifold extending between ports to define a Y-connector, the Y-connector positioned to combine blood received from a first cannula extending into the superior vena cava and blood received from a second cannula extending into the inferior vena cava for delivery to a pliable bulb syringe;
wherein the extracardiac pathway is established by cannulating the superior and inferior vena cava and the pulmonary artery of the beating heart; and
wherein the step of maintaining a substantially mechanically unobstructed flow of blood to the lungs includes the steps of:
providing the pliable bulb syringe having a reservoir capacity preselected according to a specific body characteristic of the patient,
providing a one-way valve positioned to prevent a backflow of blood from the pulmonary artery to the bulb syringe when a third cannula is operatively positioned within the pulmonary artery,
positioning the bulb syringe within the extracardiac pathway, and
iteratively compressing the bulb syringe to control the blood flow to the lungs of the patient when blood flow to the lungs of the patient undergoing the cardiac procedure is insufficient.

24. A method as defined in claim 23,
wherein the first cannula comprises a first distal portion having a first axis and a second proximal portion having a second axis oriented approximately perpendicular to the first axis of the first cannula to define a first right-angled cannula;
wherein the second cannula comprises a first distal portion having a first axis and a second proximal portion having a second axis oriented approximately perpendicular to the first axis of the second cannula to define a second right-angled cannula; and
wherein the extracardiac pathway is at least partially provided by the two right-angled cannulae when operatively positioned in the respective superior and inferior vena cavae and by a third elongate cannula defining a third straight cannula when operatively positioned in the pulmonary artery.

25. A method as defined in claim 24,
wherein the step of providing a pliable bulb syringe includes the step of applying an anticoagulant coating within the bulb syringe to thereby reduce blood clotting potential;
wherein a blood flow capacity of the first cannula, the second cannula, and the third cannula, and a blood reservoir volume of the bulb syringe, are each preselected according to a defined body characteristic of the patient;
wherein a multi-port connector defining a three-way connector is connected adjacent an outlet of the bulb syringe; and
wherein the method farther comprises the step of inserting medications and liquid fluid into the extracardiac pathway through the three-way connector.
